SEQUENCE LISTING (3 sequences)

SEQ ID NO: 1 - DNA, 32 bp, Artificial Sequence
  Synthetic
actttgcgttcgggctgggactggattggcgg

SEQ ID NO: 2 - DNA, 22 bp, Artificial Sequence
  Synthetic
cccccctgaaacgcaagcccga

SEQ ID NO: 3 - DNA, 23 bp, Artificial Sequence
  Synthetic
ccctgacctaaccgccccccccc

(19) United States
(12) Patent Application Publication
(10) Pub. No.: US 2014/0349305 A1
(43) Pub. Date: Nov. 27, 2014

(54) SYSTEMS AND METHODS FOR ONE OR MORE OF DETECTING, ISOLATING, IDENTIFYING, TRANSPORTING AND QUANTIFYING A TARGET ANALYTE IN A FLUID

(71) Applicants: Jessica O. Winter, Columbus, OH (US); Kalpesh D. Mahajan, Columbus, OH (US); Gang Ruan, Gahanna, OH (US)

(72) Inventors: Jessica O. Winter, Columbus, OH (US); Kalpesh D. Mahajan, Columbus, OH (US); Gang Ruan, Gahanna, OH (US)

(21) Appl. No.: 13/900,924

(22) Filed: May 23, 2013

(51) Int. Cl. G01N 33/543; G01N 33/58; C12Q 1/68

(52) U.S. Cl. CPC G01N 33/54333; C12Q 1/6886; G01N 33/587
USPC 435/7.1; 436/501

(57) ABSTRACT

A system for one or more of detecting, isolating, identifying, transporting, and quantifying a target analyte in a fluid suspected of containing the target analyte. The system includes a magnetic nanocomposite having one or more selectively magnetic nanoparticles in a first nanocontainer, and a first binding moiety linked to the first nanocontainer and adapted to specifically bind the target analyte, and a luminescent nanocomposite having one or more selectively luminescent nanoparticles in a second nanocontainer, and a second binding moiety linked to the second nanocontainer and adapted to specifically bind the target analyte. The magnetic nanocomposite and the luminescent nanocomposite do not specifically bind to each other in the absence of the target analyte. The first and second binding moieties may be adapted to simultaneously bind the target analyte to form a magnetic luminescent nanoassembly.

### STATEMENT REGARDING FEDERAL FUNDING

This invention was made with government support under grant numbers CBET-0707969, CMMI-0900377, EEC-0914790 and DMR-0820414, each awarded by the National Science Foundation. The United States government has certain rights in the invention.

### BACKGROUND

Several ultrasensitive nanotechnology-based diagnostic assays capable of detecting attomolar (10⁻¹⁸ M, or ˜100,000 molecules/L) or even single-molecule (10⁻²³) concentrations of biomarkers have been developed. The presence of a target molecule can induce a conformational change in probe molecules, a change in an optical or electrical signal, or nanoparticle aggregation. These changes can be amplified and converted into detectable signals such as fluorescence or a voltage change. If these detectable signals are additive, the assay can have a quantitative capability, which is critical for monitoring disease progression and response to therapy.

Most existing assays are multistep processes, involving sample enrichment, target capture, amplification (e.g., via polymerase chain reaction [PCR]), and signal detection. Although the molecules examined are all biological, they may differ dramatically in their structure and method of detection. Thus, most existing assays focus on a single type of biomarker.

Whereas multiplexed detection of molecules of the same type is challenging (e.g., by gene chips or proteomics arrays), detecting of multiple types of molecules is extremely difficult. For example, flow cytometry can detect molecules on the cell surface (and interior molecules if the cell is permeable) such as proteins or lipids, but it typically cannot independently identify miRNA or DNA expression levels and must be coupled with another assay to do so. Thus, miRNA detection is typically performed using PCR amplification followed by biochemical analysis, which would not detect concomitant protein expression. Moreover, this PCR amplification/biochemical analysis technique is largely unable to separate detected molecular targets for further analysis, modification, or manipulation.

The ability to manipulate molecules is an important component of the nanoengineering of molecular structures and small-scale synthesis (e.g., supramolecular chemistry). A need exists for improved technology for the detection and separation of biomarkers to identify and validate predictive biomarkers, which will aid in the personalization of treatments and the development of novel therapeutics. A need exists for a single, one-pot assay for detection, isolation, identification, transportation and quantification of molecular and target analytes.

### SUMMARY

This disclosure provides systems and kits for one or more of detecting, isolating, identifying, transporting, and quantifying a target analyte in a fluid suspected of containing the target analyte. The systems and kits comprise a magnetic nanocomposite comprising one or more selectively magnetic nanoparticles in a first nanocontainer, and a first binding moiety linked to the first nanocontainer and adapted to specifically bind the target analyte; and a luminescent nanocomposite comprising one or more selectively luminescent nanoparticles in a second nanocontainer, and a second binding moiety linked to the second nanocontainer and adapted to specifically bind the target analyte. The magnetic nanocomposite and the luminescent nanocomposite do not specifically bind to each other in the absence of the target analyte, and the first and second binding moieties are adapted to simultaneously bind the target analyte to form a magnetic-luminescent nanoassembly.

This disclosure also provides a magnetic-luminescent nanoassembly comprising: an analyte; a magnetic nanocomposite comprising one or more selectively magnetic nanoparticles in a first nanocontainer, and a first binding moiety specifically bound to the analyte; and a luminescent nanocomposite comprising one or more selectively luminescent nanoparticles in a second nanocontainer, and a second binding moiety specifically bound to the analyte. The magnetic nanocomposite and the luminescent nanocomposite do not specifically bind to each other.

This disclosure also provides methods for one or more of detecting, isolating, identifying, transporting, and quantifying a target analyte in a fluid suspected of containing the target analyte. The methods comprise: adding to the fluid a magnetic nanocomposite comprising one or more selectively magnetic nanoparticles in a first nanocontainer, and a first binding moiety linked to the first nanocontainer and adapted to specifically bind the target analyte; adding to the fluid a luminescent nanocomposite comprising one or more selectively luminescent nanoparticles in a second nanocontainer, and a second binding moiety linked to the second nanocontainer and adapted to specifically bind the target analyte. The magnetic nanocomposite and the luminescent nanocomposite do not specifically bind to each other in the absence of target analyte, and the first and second binding moieties are adapted to simultaneously bind the target analyte to form a magnetic-luminescent nanoassembly. The methods further comprise applying a magnetic field to at least a portion of the fluid with a magnetic manipulator.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing a system for detecting, isolating, identifying, transporting, and/or quantifying a target analyte, according to aspects of the present invention.

FIG. 2 is a series of images showing the isolation, translation and detection of a single-stranded DNA sequence of the p53 oncogene (“p53 DNA”) using a system according to the present disclosure.

FIG. 3 is a series of images showing the isolation and detection of avidin using a system according to the present disclosure.

FIG. 4 is a series of images showing a) directed movement of fluorescent-magnetic nanoassemblies comprising an avidin analyte (red) and Brownian motion of p53 DNA bound to a fluorescent nanocomposite (green) in the absence of any magnetic nanocomposite that selectively binds p53 DNA; b) directed movement of fluorescent-magnetic nanoassemblies comprising p53 DNA (green); and c) simultaneous directed movement of first fluorescent-magnetic nanoassemblies containing avidin (red) and second fluorescent-magnetic nanoassemblies comprising p53 DNA (green).

FIG. 5 is a schematic showing the concentration dependent formation of magnetic-luminescent nanoassemblies comprising fluorescent and magnetic nanocomposites that each having a plurality of binding moieties specific for a target analyte.

FIG. 6 is a graph of a calibration curve showing the relationship between p53 DNA concentration and fluorescence intensity.

### DETAILED DESCRIPTION

The present disclosure is not limited in its application to the specific details of construction, arrangement of components, or method steps set forth herein. The compositions and methods disclosed herein are capable of being made, practiced, used, carried out and/or formed in various ways. The phraseology and terminology used herein is for the purpose of description only and should not be regarded as limiting. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification, and no structures shown in the drawings, should be construed as indicating that any non-claimed element is essential to the practice of the invention. The use herein of the terms “including,” “comprising,” or “having,” and variations thereof, is meant to encompass the items listed thereafter and equivalents thereof, as well as additional items. Unless specified or limited otherwise, the terms “mounted,” “connected,” “supported,” and “coupled” and variations thereof encompass both direct and indirect mountings, connections, supports, and couplings. Further, “connected” and “coupled” are not restricted to physical or mechanical connections or couplings.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities.

This disclosure provides systems, methods and kits for one or more of detecting, isolating, identifying, transporting, and quantifying a target analyte in a fluid suspected of containing the target analyte. This disclosure also provides magnetic-luminescent nanoassemblies, and methods of making and using the same.

In principle, the target analyte may be any composition capable of being simultaneously and selectively bound by at least one magnetic nanocomposite and at least one luminescent nanocomposite. Target analytes may include small molecules, macromolecules, and macrostructures, including, but not limited to, proteins, carbohydrates, fats, nucleic acids, cells and cellular structures, such as organelles. In some embodiments, the target analyte may be a biomarker. Biomarkers include molecules that can be objectively measured as an indicator of normal biological processes, disease conditions, or responses to drug treatment. Some biomarkers may be soluble in aqueous media, whereas others may not. Biomarkers may be used to identify the natural disease process, and also may indicate the potential clinical benefit of a specific treatment.

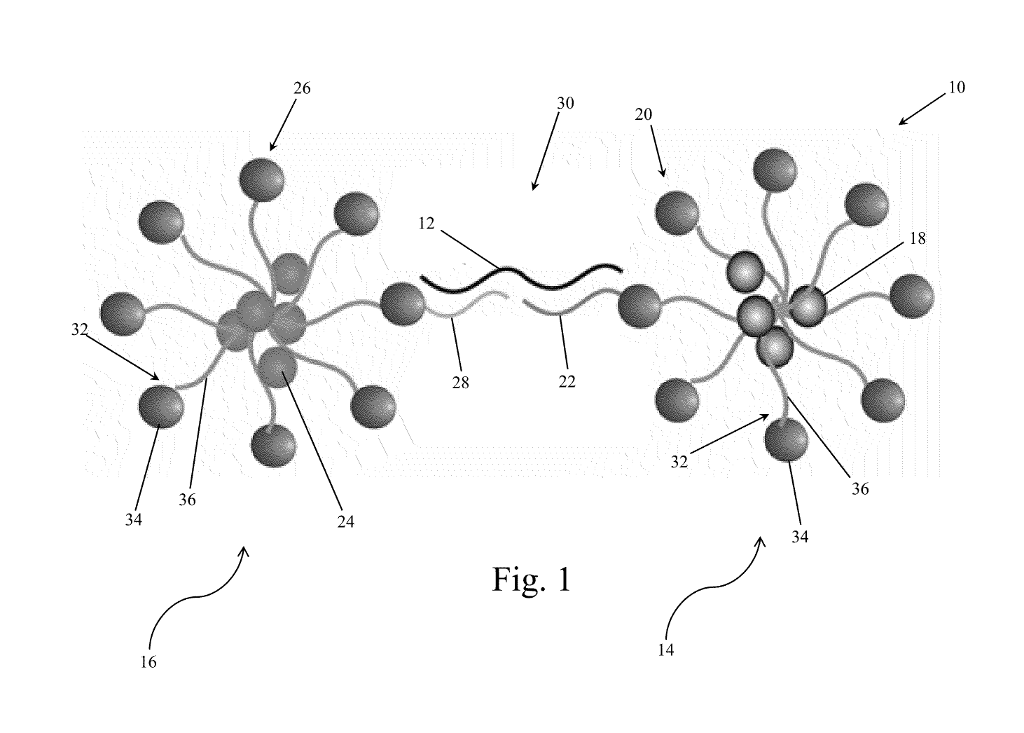

FIG. 1 shows an exemplary system 10 for detecting isolating, identifying, transporting, and quantifying a target analyte 12. System 10 may include a magnetic nanocomposite 14 and a luminescent nanocomposite 16, each adapted to specifically bind to the target analyte 12. As used herein, the terms specific binding, specifically bind, or any variation thereof, shall mean a binding interaction that is saturable and selective. The magnetic nanocomposite 14 and the luminescent nanocomposite 16 may be adapted to simultaneously bind the target analyte 12 to form a magnetic-luminescent nanoassembly 30, but to not specifically bind to each other in the absence of the target analyte 12.

The magnetic nanocomposite 14 may include one or more selectively magnetic nanoparticles 18 in a first nanocontainer 20, and a first binding moiety 22 linked to the first nanocontainer 20 and adapted to specifically bind the target analyte 12. The term selectively magnetic, as used herein, means that the magnetic nanoparticles 18 are substantially non-magnetic in the absence of a magnetic field, but may be manipulated using a magnetic field. As such, the magnetic nanoparticles 18 may comprise one or more paramagnetic or superparamagnetic materials, including, but not limited to, iron, cobalt, nickel, and carbides, nitrides, sulfides, phosphides and oxides thereof. For example, the magnetic nanoparticles 18 may comprise superparamagnetic iron oxide (e.g., γ-Fe₂O₃ or Fe₃O₄). In some embodiments, the magnetic nanoparticles 18 may include multiple layers, including a core and one or more shells, where at least one of the layers is selectively magnetic. In some embodiments, the magnetic nanoparticles 18 may be surface functionalized. The magnetic nanoparticles 18 may range in size from about 0.1 nm to about 50 nm. Examples of magnetic nanoparticles 18 that may be used to form magnetic nanocomposites according to this disclosure may include, but are not limited to, those disclosed in U.S. Pat. Nos. 8,409,341, 8,383,085, 8,343,577, 8,323,618, 8,318,093, 8,303,838, 8,277,581, 7,556,863, 7,128,891, 7,029,514, 6,962,685, 6,767,635, 6,548,264, 5,783,263, 5,427,767, 4,554,088, and 4,452,773, the complete disclosures of which are herein incorporated reference.

Each magnetic nanocomposite 14 may include one or more different species of magnetic nanoparticles 18, where each species of nanoparticle has a different chemical composition, structure and/or set of physical properties. The magnetic properties of each magnetic nanocomposite 14, in turn, depend on the particular magnetic nanoparticles 18 from which the nanocomposite is formed. As such, different species of magnetic nanocomposites 14 may be used to form a plurality of different species of magnetic nanocomposites 14, each having a unique magnetic “fingerprint”. Likewise, a system 10 may comprise a plurality of species of magnetic nanocomposites 14, where each species has a different magnetic “fingerprint” and is independently adapted to bind to a selected target analyte. For example, a system may include a first magnetic nanocomposite having a first magnetic fingerprint, and a second magnetic nanocomposite having a second magnetic fingerprint, where the first and second magnetic nanocomposites are either adapted to bind to the same target analyte or to different target analytes.

The first nanocontainer 20 may comprise any container adapted to substantially encapsulate the one or more magnetic nanoparticles 18. It may be desirable for the magnetic nanocomposite 14 to include a plurality of magnetic nanoparticles 18 maintained in close proximity to one another for purposes of tuning the properties of the nanocomposite 14. Moreover, some magnetic nanoparticles 18 may be slightly or substantially insoluble in the fluid suspected of containing the target analyte and/or may tend to aggregate in the fluid. For example, some magnetic nanoparticles may be hydrophobic and thus may be slightly or substantially insoluble in an aqueous solvent. The first nanocontainer 20 may be adapted to maintain the desired plurality of magnetic nanoparticles 18 in close proximity to one another and/or to solubilize the magnetic nanoparticles 18. In some embodiments, the first nanocontainer 20 may include a plurality of self-assembling subunits. For example, as shown in FIG. 1, the first nanocontainer 20 may be a micelle comprising an amphiphile 32 including a hydrophilic moiety 34 and a hydrophobic moiety 36. Suitable amphiphiles may include, but are not limited to, amphiphilic block copolymers, peptide amphiphiles, lipid amphiphiles, and combinations thereof. For example, amphiphilic block copolymers may include, but are not limited to, poly(styrene-b-ethylene glycol), poly(ε-caprolactone-b-ethylene glycol), poly(ethylene glycol-b-distearoyl phosphatidylethanolamine), and combinations thereof. Peptide amphiphiles may include, but are not limited to, palmitoyl-VVAAEE-NH₂, palmitoyl-VVAAEEGIKVAV-COOH, palmitoyl-VVAAEEEEGIKVAV-COOH, and combinations thereof. Lipid amphiphiles may include, but are not limited to, phospholipids, glycolipids, cholesterol, fatty acids, and combinations thereof.

It should be appreciated that the size and shape of the magnetic nanocomposite 18 may depend on the composition of the first nanocontainer 20 (e.g., the type of amphiphile utilized). For example, nanocontainers 20 formed of poly(styrene-b-ethylene glycol) having a molecular weight of 3800-b-6500 Daltons may have diameters averaging about 25 nm as confirmed by dynamic light scattering (DLS), transmission electron microscopy (TEM) and single particle tracking (SPT). In contrast, nanocontainers 20 formed of poly(styrene-b-ethylene glycol) having a molecular weight of 9500-b-18000 Daltons may have diameters averaging about 40 nm as confirmed by DLS, TEM and SPT. Other amphiphiles, such as distearoyl phosphatidylethanolamine-co-polyethylene glycol 2,000 (DSPE-PEG) may form micelles having diameters averaging about 15 nm as confirmed by DLS, TEM and SPT, with a core diameter of about 6.5 nm. Thus, when engineering nanocomposites for specific applications requiring a particular size of nanocomposite, the size can be controlled by selecting an appropriate nanocontainer. Moreover, amphiphilic block copolymers are particularly advantageous because these materials generally have a relatively long hydrophobic segment. The longer hydrophobic segment allows for the formation of amphiphilic micelles having a larger hydrophobic core so that multiple and diverse types of nanoparticles can be encapsulated within the micelle, while at the same time remaining small enough to be particularly useful in various diverse applications.

The exterior surface of the first nanocontainer 20 may be adapted to be linked to the first binding moiety 22. For example, the first nanocontainer 20 may include an exterior surface having at least one functional group adapted to react with a functional group on the first binding moiety 22 to form a covalent bond, as discussed in more detail below. The first nanocontainer 20 also may be adapted to have an extremely high binding affinity for the first binding moiety 22, and/or to include a functional group having an extremely high affinity for the first binding moiety. For example, in the case of nanocontainers formed of amphiphiles, the hydrophilic moiety 34 may be functionalized to react or bind tightly to the first binding moiety. Linking means for attaching binding moieties to the surface of nanomaterials are well known in the art, and any such suitable means may be used in accordance with the present disclosure.

The first binding moiety 22 may be linked to the first nanocontainer 20, and adapted to specifically bind the target analyte 12. Depending on the target analyte 12, the first binding moiety 22 may include a peptide, a polypeptide, a protein (e.g., an antibody), a nucleic acid, an oligonucleotide, a polysaccharide, and combinations thereof, or any other binding moiety having a high affinity for the target analyte. As discussed above, the first binding moiety 22 may be linked to the first nanocontainer 20 using any suitable means. For example, the first binding moiety 22 may be covalently linked to the first nanocontainer 20 using carbodiimide chemistry or NHS-ester crosslinker chemistry, among numerous other linking chemistries. The first binding moiety also may be linked to the nanocontainer using non-covalent means, including, but not limited to, utilizing the strong affinity between streptavidin and biotin. In some embodiments, the first binding moiety 22 may be linked to a component of the first nanocontainer 20 (e.g., to the hydrophilic moiety of an amphiphile) prior to assembly (e.g., self-assembly) of the nanocontainer 22. In other embodiments, the first binding moiety 22 may be linked to the first nanocontainer 20 after the nanocontainer has been formed.

In some embodiments, the magnetic nanocomposite 14 may include a plurality of first binding moieties 22 linked to the first nanocontainer 20. Each of the plurality of first binding moieties may be the same or different. For example, in the case of nanocontainers formed of amphiphiles, the overall quantity or concentration of binding moieties linked to the nanocontainer may be varied, for example, by adjusting the relative concentrations of amphiphiles that have been linked to binding moieties and amphiphiles that have not been linked to binding moieties.

The luminescent nanocomposite 16 may include one or more selectively luminescent nanoparticles 24 in a second nanocontainer 26, and a second binding moiety 28 linked to the second nanocontainer 28 and adapted to specifically bind the target analyte 12. The term selectively luminescent, as used herein, means that the luminescent nanoparticles 24 emit light upon selective excitation using an external energy source. Selectively luminescent nanoparticles 24 may include, but are not limited to, chemiluminescent, electroluminescent, and photoluminescent nanoparticles, among others. In some embodiments, the selectively luminescent nanoparticles 24 are photoluminescent nanoparticles, such as fluorescent nanoparticles. Exemplary luminescent nanoparticles 24 may include quantum dots, including semiconducting quantum dots, and carbon dots. In some embodiments, the selective luminescence of the luminescent nanoparticles 24 may be remotely triggered, for example by an external electromagnetic radiation source, such as a solid state light source, a high intensity light source, or a laser, among others. The emission spectra of the luminescent nanoparticles 24 may be measured with a light detector according to known methods. The luminescent nanoparticles 24 may have sizes ranging from about 0.5 nm to about 70 nm in diameter. Examples of suitable luminescent nanoparticles 24 that may be used to form the luminescent nanocomposites 16 of this disclosure, and methods of making the same, may include, but are not limited to, those disclosed in U.S. Pat. Nos. 8,287,761, 8,003,166, 7,790,473, 7,306,823, 7,282,732, 7,172,791, 6,815,064, 6,699,723, 6,048,616, and 5,990,479, the complete disclosures of which are herein incorporated reference.

Each luminescent nanocomposite 16 may include one or more different species of luminescent nanoparticle 24, where each species of nanoparticle has a different chemical composition, structure and/or set of physical properties. The luminescent properties of each luminescent nanocomposite 16, in turn, depend on the particular luminescent nanoparticles 24 from which the luminescent nanocomposite is formed. As such, different species of luminescent nanoparticles 24 may be used to form a plurality of different species of luminescent nanocomposites 16, each having a unique luminescent excitation and emission “fingerprint”. Likewise, a system 10 may comprise a plurality of species of magnetic nanocomposites 14, where each species has a different luminescent “fingerprint” and is independently adapted to bind to a selected target analyte. For example, a system may include a first luminescent nanocomposite having a first luminescent fingerprint, and a second luminescent nanocomposite having a second luminescent fingerprint, where the first and second luminescent nanocomposites are either adapted to bind to the same target analyte or to different target analytes.

The second nanocontainer 26 may comprise any container adapted to substantially encapsulate the one or more luminescent nanoparticles 24. The second nanocontainer 26 functions similarly to, and may be formed of substantially the same materials as, the first nanocontainer 20, discussed above. The exterior surface of the second nanocontainer 26 may be adapted to be linked to the second binding moiety 28, and the second binding moiety may be linked to the second nanocontainer 26 in any suitable manner, such as is similarly described above with respect to the first binding moiety 22 and the first nanocontainer 20.

The second binding moiety 28 may be adapted to specifically bind to the target analyte 12, such that both the first binding moiety 22 and the second binding moiety 28 may simultaneously bind the target analyte 12 to form a magnetic-luminescent nanoassembly 30. The first and second binding moieties associated with a particular magnetic-luminescent nanoassembly 30 must, therefore, have different compositions so that they can simultaneously bind to the same analyte. But otherwise, the second binding moiety 28 may function similarly to, and may be formed of substantially the same materials as are described above for the first binding moiety 22.

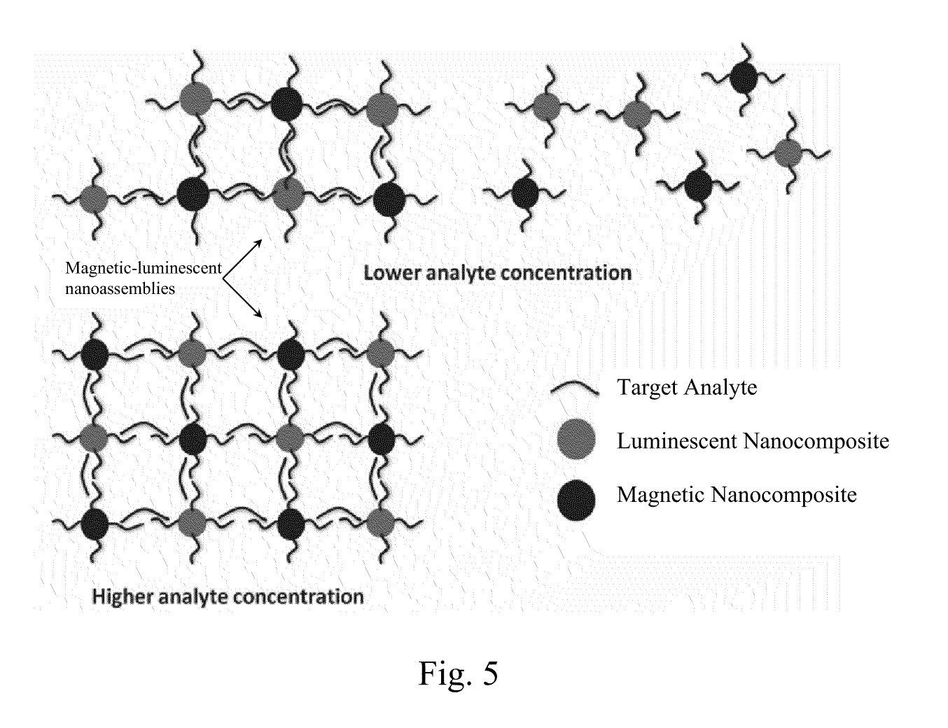

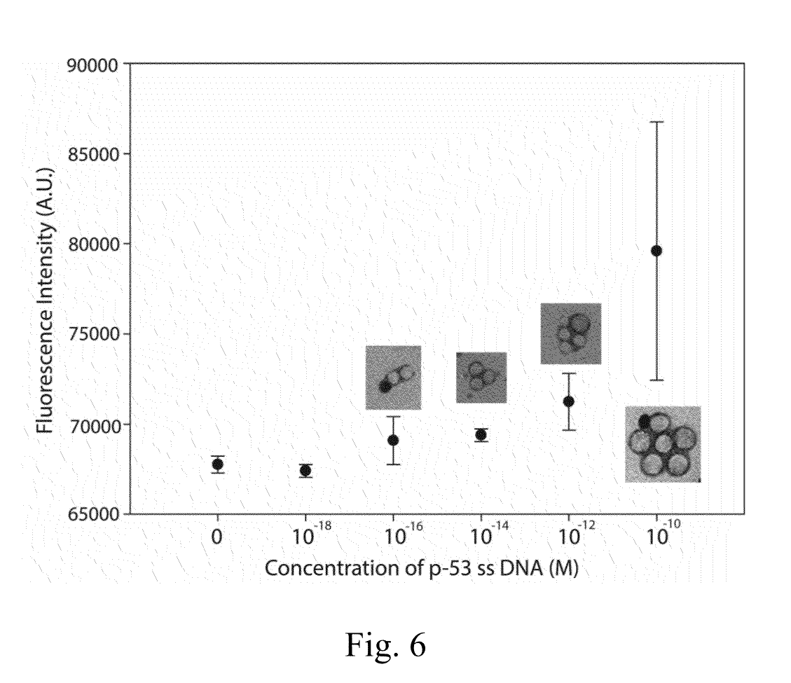

As indicated above, some magnetic and luminescent nanocomposites each may include a plurality of first and second binding moieties, respectively. When such nanocomposites are added to a solution containing target analyte, each magnetic and luminescent nanocomposite molecule may bind to a plurality of analyte molecules, thus forming a magnetic-luminescent nanoassembly comprising a plurality of magnetic nanocomposite molecules, a plurality of luminescent nanocomposite molecules, and a plurality of analyte molecules, as is best shown in FIG. 5. As shown in FIGS. 5 and 6, the overall size of these magnetic-luminescent nanoassemblies, as well as the magnitude of the luminescent emission (e.g., fluorescence) generated by the nanoassemblies, is dependent on the concentration of analyte. This dependence permits for the quantification of the concentration of analyte based on the magnitude of the luminescent signal, such as via the use of a standard calibration curve.

The magnetic and luminescent nanocomposites described above may be formed by any suitable method, including, but not limited to, the method for forming nanocomposite particles disclosed in U.S. Patent Application Pub. No. 2013/0078469, which is incorporated herein in its entirety by reference. The magnetic and luminescent nanocomposites formed by such methods may have sizes ranging from about 5 nm to about 1,000 nm in diameter. As shown in FIG. 1, the magnetic nanocomposite 14 and the luminescent nanocomposite 16 can, in turn, be used to form the magnetic-luminescent nanoassembly 30 simply by mixing the magnetic and luminescent nanocomposites together in a solution with the target analyte. The magnetic-luminescent nanoassembly 30 permits for the detection, isolation, identification, transportation and quantification of the target analyte 12.

In some cases, it may desirable to detect, isolate, identify, transport and/or quantify a plurality of distinct target analytes that may be present in the same fluid. Systems for performing such experiments may comprise a first magnetic nanocomposite and a first luminescent nanocomposite, each adapted to bind to a first analyte to form a first magnetic-luminescent nanoassembly, and a second magnetic nanocomposite and a second luminescent nanocomposite, each adapted to bind to a second analyte to form a second magnetic-luminescent nanoassembly. The first and second magnetic-luminescent nanoassemblies each may be selected to have different magnetic and/or fluorescent properties that permit for distinguishing the first and second magnetic-luminescent nanoassemblies from each other.

The systems 10 disclosed herein further may comprise one or more of a magnetic manipulator, a light source for exciting the luminescent nanoparticles, and a light detector for detecting light emitted by the luminescent nanoparticles. Magnetic manipulators may comprise any structure capable of physically manipulating the magnetic nanocomposites, including, but not limited to, a magnetic needle or a magnetic nanoconveyor. Magnetic nanoconveyors may be fabricated in a variety of shapes (e.g., wires, disks, etc.) using standard microfabrication techniques and electron beam lithography on a silicon substrate. For example, magnetic nanoconveyors suitable for use in the systems disclosed herein may be made according to methods disclosed in Vieira, G., T. Henighan, A. Chen, A. J. Hauser, F. Y. Yang, J. J. Chalmers, and R. Sooryakumar, Magnetic Wire Traps and Programmable Manipulation of Biological Cells. Physical Review Letters, 2009. 103(12): p. 128101 and Henighan, T., A. Chen, G. Vieira, A. J. Hauser, F. Y. Yang, J. J. Chalmers, and R. Sooryakumar, Manipulation of Magnetically Labeled and Unlabeled Cells with Mobile Magnetic Traps. Biophysical Journal, 2010. 98(3): p. 412-417, each of which is hereby incorporated by reference in its entirety.

The force generated by the magnetic manipulator must be sufficient to overcome Brownian motion of the target analyte, which can be considerable if the size of the target analyte is small.

The various systems disclosed herein may be provided in the form of kits that further include instructions for using the systems to perform one or more of detecting, isolating, identifying, transporting, and quantifying the target analyte in the fluid. The instructions may comprise identification of the analyte to which any nanocomposites contained in the system bind, identification of the binding moiety of any nanocomposites contained in the system, or a combination thereof. The instructions may comprise information relating to the magnetic or luminescent properties of any nanocomposites contained in the system. The instructions may comprise information relating to the properties required of a fluid for proper functioning of the system. The instructions may comprise a means of correlating a luminescence measurement with the identification or quantification of an analyte.

This disclosure further provides methods for one or more of detecting, isolating, identifying, transporting, and quantifying a target analyte in a fluid suspected of containing the target analyte. The methods may comprise adding to the fluid a magnetic nanocomposite, adding to the fluid a luminescent nanocomposite, and applying a magnetic field to at least a portion of the fluid with a magnetic manipulator. The methods may comprise at least one of isolating the target analyte at, or transporting the target analyte to, a location by manipulating the magnetic-luminescent nanoassembly with the magnetic field. The methods also may comprise at least one of detecting, identifying or quantifying the target analyte by selectively inducing luminescence of the luminescent nanoparticles, and observing at least one of the existence or intensity of luminescence from the magnetic-luminescent nanoassembly. Finally, the methods may comprise quantifying the target analyte by manipulating the magnetic-luminescent nanoassembly with the magnetic field, and measuring the amount of magnetic mobility. It should be appreciated that quantification of the target analyte using either luminescence intensity or magnetic mobility may be facilitated by the formation of a standard calibration curve. Moreover, the simultaneous use of both luminescence intensity and magnetic mobility to measure the concentration of analyte may provide a built-in quality control check to ensure that the measurements match up.

It should be appreciated that a primary advantage of the methods disclosed herein resides in the fact that only nanocomposites having a magnetic component and a luminescent component (i.e., magnetic-luminescent nanoassemblies) are capable of both being manipulated by a magnetic manipulator and luminescing. Magnetic-luminescent nanoassemblies thus may be manipulated to a selected position by a magnetic manipulator until the localized concentration of magnetic-luminescent nanoassemblies is high enough to be able to generate a detectable luminescent signal at that position. In contrast, nanocomposites and nanoassemblies comprising only the magnetic nanocomposite may be manipulated by the magnetic manipulator, but will not luminesce in the absence of the luminescent nanocomposite. Similarly, nanocomposites and nanoassemblies comprising only the luminescent nanocomposite will not be capable of being manipulated by the magnetic manipulator, and therefore will not be capable of being concentrated at a selected position to a sufficient concentration for generating a detectable signal.

In some embodiments, the methods of this disclosure may be performed without damaging or destroying the target analyte, so that the target analyte is available for further analysis or processing.

### EXAMPLES

### Example 1

### DNA Isolation and Detection

A 32-nucleotide single-stranded DNA sequence (SEQ ID NO 1: ACTTTGCGTTCGGGCTGGGACTGGATTGGCGG) of the p53 oncogene (“p53 DNA”) was prepared in aqueous solution at a concentration of about 10⁻¹⁶ M.

100 μL quantum dots (QDs) (λₑₘ, 545 nm cat No. Q21791, λₑₘ, 605 nm Cat No. Q21701, Life Technologies, Inc.) in decane as supplied by the manufacturer were flocculated in a mixture of 150 μL isopropanol and 300 μL methanol and then re-suspended in chloroform at a concentration of 0.1 μM.

Superparamagnetic Iron Oxide Nanoparticles (SPIONs) (5 nm Cat No. SOR-05-50 Ocean nanotech) were dissolved in chloroform at a concentration of 3.45 μM.

The amphiphilic block copolymer carboxyl terminated poly(styrene-b-ethylene oxide) PS(9500)-b-PEO(18000) (Cat No. P5755-SEOCOOH, Polymer Source Inc.) was dissolved in chloroform at a concentration of 36.4 μM.

Micelles containing QDs were formed by mixing the QD solution formed above (100 μl, 0.1 μM) with the carboxylated amphiphilic polymer solution formed above (10 μl, 36.4 μM) and 100 μL of chloroform, and then dispersing the 210 μL organic mixture in 800 μL of 5 mg/ml aqueous poly(vinyl alcohol) (PVA, 13,000-23,000 Dalton, 87-89% hydrolyzed, cat no. 363170 Aldrich) solution to obtain an emulsion. The chloroform was evaporated from this emulsion to obtain a clear and transparent micelle dispersion where the micelles encapsulate QDs (QD-micelles).

Micelles containing SPIONs were formed by mixing the SPION solution formed above (100 μl, 3.45 μM) with the carboxylated amphiphilic polymer solution formed above (10 μl, 36.4 μM) and 100 μL of chloroform, and then dispersing the 210 μL organic mixture in 800 μL of 5 mg/ml aqueous poly(vinyl alcohol) (PVA, 13,000-23,000 Dalton, 87-89% hydrolyzed, cat no. 363170 Aldrich) solution to obtain an emulsion. The chloroform was evaporated from this emulsion to obtain a clear and transparent micelle dispersion where the micelles encapsulate SPIONs (SPION-micelles).

Luminescent nanocomposites were formed by functionalizing the QD-micelles with amine terminated single stranded DNA binding moieties complimentary to a portion of the p53 DNA sequence (SEQ ID NO 2: NH₂C₆-TGAAACGCAAGCCCGA) (custom made, Sigma). Specifically, the amine terminated single stranded DNA having the sequence SEQ ID NO 2 was conjugated to the carboxylated QD-miscelles through N-(3-Demethylaminopropyl)-N′-ethylcarbodiimide hydrochloride (EDC) chemistry. The QD miscelle solution at a pH of 7.5 was mixed with EDC (Thermo Scientific), sulpho-NHS (Thermo Scientific) and the single stranded DNA at a molar ratio of HOOC—PS-PEO:EDC:sulpho-NHS:ssDNA of 1:1000:2500:100. This reaction mixture was stirred overnight at room temperature. The luminescent nanocomposites (i.e., DNA functionalized-QD-miscelles) were then dialyzed against deionized water to remove unreacted reagents.

Magnetic nanocomposites were similarly formed by functionalizing the SPION-micelles with amine terminated single stranded DNA binding moieties complimentary to a portion of the p53 DNA sequence (SEQ ID NO 3: CCCTGACCTAACCGCC-C₇NH₂) (custom made, Sigma). SEQ ID NOs 2 and 3 do not bind to each other, and each is adapted to simultaneously bind the p53 DNA target analyte. The amine terminated single stranded DNA having the sequence SEQ ID NO 3 was conjugated to the carboxylated SPION-miscelles in the same way that SEQ ID NO 2 was conjugated to the carboxylated QD-miscelles.

Magnetic nanoconveyors were formed from Co_{0.5}Fe_{0.5} nanowires according to the method described in Vieira, et al., Magnetic Wire Traps and Programmable Manipulation of Biological Cells. Physical Review Letters, 2009. 103(12): p. 128101, the entire disclosure of which is herein incorporated by reference. Zigzag wires with a vertex-to-vertex distance of 4 μm were patterned onto silicon substrates with electron beam lithography. The layers of e-beam resist (methylmethacrylate and polymethyl methacrylate) were spin coated, exposed and developed, followed by magnetron sputter deposition of 40 nm of Co_{0.5}Fe_{0.5}. The resultant Co_{0.5}Fe_{0.5} wires were momentarily magnetized by an external field (˜1,000 Oe), which was then removed to allow magnetization to relax along the length of the wire. This ensured that wire magnetization would alternate at each leg, creating domain walls at each vertex, and that the domain wall profile, either head-to-head or tail-to-tail, alternates at neighboring vertices. The substrates were coated with a 1 nm seed layer of permalloy and a 5 nm layer of gold by magnetron sputtering. The gold surface was cleaned by UV ozone treatment for ˜10 min and submerged in a 1 mM polyethylene glycol (PEG)-SH (molecular weight 5000, Laysan Bio, Arab, Ala., USA) solution in ethyl alcohol for at least 1 hr, allowing a PEG monolayer to form. The surface was then rinsed in ethyl alcohol and deionized water and dried with air or nitrogen. This surface modification helps prevent biofouling and non-specific binding and increases the hydrophilicity of the surface.

A magnetic trapping and manipulation system was assembled comprising the patterned zigzag nanowires, two pairs of electromagnets for applying in-plane magnetic fields (H_{xy}), and a solenoid coil for applying an out-of-plane field (H_{z}). The out-of-plane field acts to strengthen, weaken, or reverse the magnetic traps. The in-plane field acts to direct the motion of a trapped particle.

The system was mounted on the stage of a reflected fluorescence microscope (Olympus BX 41). A 5 μL sample drop was placed on the substrate, which was covered with a coverslip and immersion oil. An out-of-plane field (H_{z}) of ˜100 Oe was applied upward, which allows magnetic particles to be trapped at specific locations. At selected time points, the direction of H_{z} was switched by reversing the current in the solenoid coil, moving magnetic structures between wire vertexes. Fluorescent imaging was performed using a 100× oil immersion objective (Olympus), 100 W mercury lamp, long-pass filter, and an Olympus DP70 CCD camera. Image processing and analysis was conducted using ImageJ image analysis software by combining brightfield background images showing the wire arrays with fluorescence images showing the nano-scale particles.

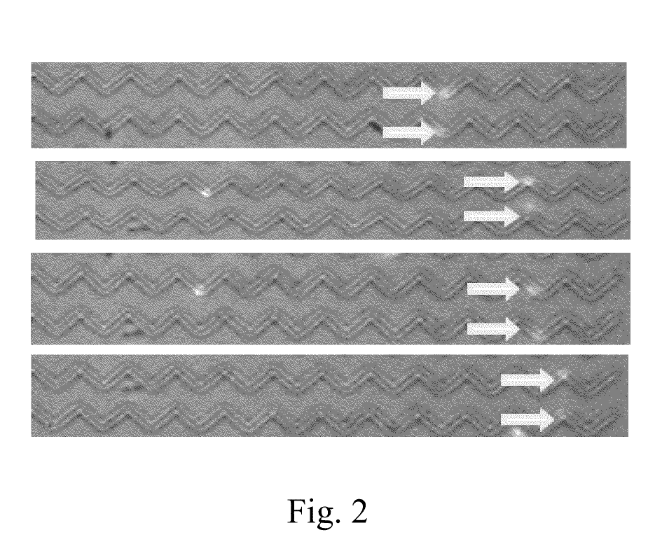

The luminescent nanocomposite and magnetic nanocomposite were added to the solution containing the p53 DNA, and the solution was exposed to the magnetic nanoconveyors. As shown in FIG. 2, a detectable luminescent signal translated along the magnetic nanoconveyors, thus demonstrating that the luminescent and magnetic nanocomposites simultaneously bound to the p53 DNA to form magnetic-luminescent nanoassemblies.

### Example 2

### Avidin Isolation and Detection

The procedure of Example 1 was generally repeated, with a few minor modifications. First, the protein avidin was selected as the target analyte instead of p53 DNA, and the binding moieties of the luminescent and magnetic nanocomposites were replaced with biotin binding moieties, which selectively bind avidin. Specifically, luminescent and magnetic nanocomposites were each independently formed by mixing the carboxylated QD-miscelle and carboxylated QD-SPION solutions, respectively, at pH 7.5 with EDC, sulpho-NHS and pentyl amine biotin (Cat. No. 21345, Thermo Scientific) at the molar ratio of HOOC—PS-PEO:EDC:sulpho-NHS:Biotin 1:1000:2500:100. These reaction mixtures were stirred overnight at room temperature. The luminescent nanocomposites (i.e., biotin functionalized-QD-miscelles) and magnetic nanocomposites (i.e., biotin functionalized SPION-miscelles) were then dialyzed against deionized water to remove unreacted reagents.

Second, Ni_{0.8}Fe_{0.2} nanodisks were used instead of the Co_{0.5}Fe_{0.5} nanowires. The nanodisks were likewise formed according to the methods described in Vieira, et al., Magnetic Wire Traps and Programmable Manipulation of Biological Cells. Physical Review Letters, 2009. 103(12): p. 128101, the entire disclosure of which is herein incorporated by reference. Ferromagnetic disks with a diameter of 4 μm were patterned onto silicon substrates with electron beam lithography. The layers of e-beam resist (methylmethacrylate and polymethyl methacrylate) were spin coated, exposed and developed, followed by magnetron sputter deposition of 40 nm of Ni_{0.8}Fe_{0.2}. The substrates were coated with a 1 nm seed layer of permalloy and a 5 nm layer of gold by magnetron sputtering. The gold surface was cleaned by UV ozone treatment for ˜10 min and submerged in a 1 mM polyethylene glycol (PEG)-SH (molecular weight 5000, Laysan Bio, Arab, Ala., USA) solution in ethyl alcohol for at least 1 hr, allowing a PEG monolayer to form. The surface was then rinsed in ethyl alcohol and deionized water and dried with air or nitrogen. This surface modification helps prevent biofouling and non-specific binding and increases the hydrophilicity of the surface.

A magnetic trapping and manipulation system was assembled comprising the nanodisks on a silicon substrate, two pairs of electromagnets for applying in-plane magnetic fields (H_{xy}), and a solenoid coil for applying an out-of-plane field (H_{z}). The out-of-plane field acts to strengthen, weaken, or reverse the magnetic traps. The in-plane field acts to magnetize disks to generate traps on their periphery and to direct the motion of a trapped particle.

The system was mounted on the stage of a reflected fluorescence microscope (Olympus BX 41). A 5 μL sample drop was placed on the substrate, which was covered with a coverslip and immersion oil. An out-of-plane field (H_{z}) of ˜100 Oe was applied upward, which allows magnetic particles to be trapped at specific locations. At selected time points, the in-plane field was rotated, maneuvering the magnetic structures around the disk periphery. Fluorescent imaging was performed using a 100× oil immersion objective (Olympus), 100 W mercury lamp, long-pass filter, and an Olympus DP70 CCD camera. Image processing and analysis was conducted using ImageJ image analysis software by combining brightfield background images showing the disk arrays with fluorescence images showing the nano-scale particles.

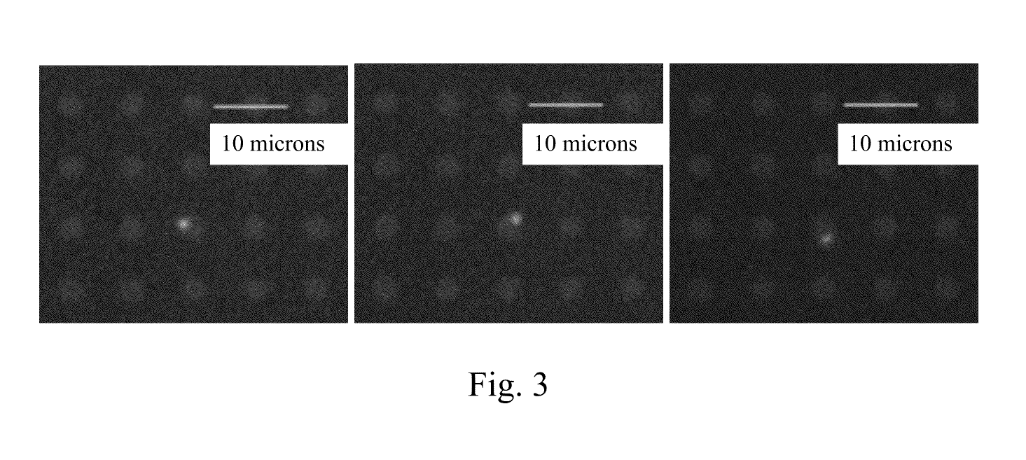

The luminescent nanocomposite and magnetic nanocomposite were added to a solution containing avidin to form a reaction solution, and the solution containing the avidin and the luminescent and magnetic nanocomposites was exposed to a varying magnetic field. As shown in FIG. 3, a detectable luminescent signal was detected and localized on one of the nanodisks, indicating that the luminescent and magnetic nanocomposites bound to the avidin to form magnetic-luminescent nanoassemblies, which were trapped by the applied magnetic field in the z-plane. The applied magnetic field was then varied in the x-y plane in order to move the magnetic-luminescent nanoassemblies from the upper left portion of the disk (left image) to the upper right portion of the disk (central image) to the lower portion of the disk (right image).

### Example 3

### DNA and Avidin Isolation, Detection, Identification and Translation

A first solution containing the magnetic-luminescent nanoassembly comprising p53 DNA (“the p53-magnetic-luminescent nanoassembly”) was prepared according to the same procedure as in Example 1. A second solution was prepared in exactly the same manner as the first solution, but without the addition of any magnetic nanocomposite. In other words, the second solution contained the p53 DNA and the luminescent nanocomposite of Example 1 (“the p53-luminscent nanoassembly”). A third solution containing the magnetic-luminescent nanoassembly comprising avidin (“the avidin-magnetic-luminescent nanoassembly”) were prepared according to the same procedure as in Example 2.

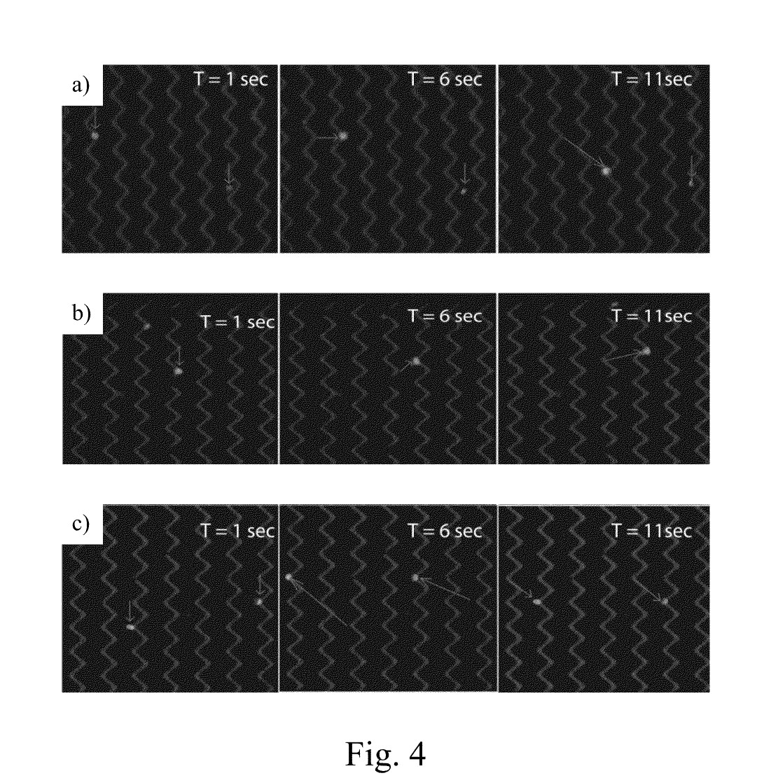

The second and third solutions were mixed together to form a fourth solution comprising both the p53-luminscent nanoassembly and the avidin-magnetic-luminescent nanoassembly. The fourth solution was exposed to magnetic nanoconveyors and a magnetic field was applied in the x-y plane from top to bottom for several seconds, then from left to right for several seconds, then from upper left to lower right for several seconds. As shown in FIG. 4(a), the varying magnetic field caused the avidin-magnetic-luminescent nanoassembly (red) to translate along the magnetic nanoconveyors in the directions of the applied magnetic field. In contrast, the p53-luminescent nanoassembly (green) failed to translate in the x-y plane with the application of the magnetic field.

After conducting the experiment shown in FIG. 4(a), a magnetic field was applied in the x-y plane until the avidin-magnetic-luminescent nanoassembly was removed from the field of view. The magnetic nanoparticles of Example 1 were then added to the fourth solution in an attempt to form a p53-magnetic-luminescent nanoassembly. A magnetic field was then applied in the x-y plane from top to bottom for several seconds, then from lower left to upper right for several seconds, then from left to right and slightly upwards for several seconds. As shown in FIG. 4(b), the varying magnetic field caused the p53-magnetic-luminescent nanoassembly (green) to translate along the magnetic nanoconveyors in the directions of the applied magnetic field.

Next, the first and third solutions prepared above were mixed together to form a fifth solution comprising both the p53-magnetic-luminscent nanoassembly and the avidin-magnetic-luminescent nanoassembly. The fifth solution was exposed to the magnetic nanoconveyors and a magnetic field was applied in the x-y plane from top to bottom for several seconds, then from lower left to upper right for several seconds, then from upper left to lower right for several seconds. As shown in FIG. 4(c), the varying magnetic field caused both the avidin-magnetic-luminescent nanoassembly (red) and the p53-magnetic-luminscent nanoassembly (green) to translate along the magnetic nanoconveyors in the directions of the applied magnetic field.

### Example 4

### DNA Concentration-Dependent Molecular Assembly and Concentration Calibration

The procedure of Example 1 was repeated to prepare a series of solutions having varying concentrations of p53 DNA and with fluorescent nanocomposites and magnetic nanocomposites that have more than one binding moiety. FIG. 5 shows a schematic representation of the magnetic-luminescent nanoassemblies formed at both lower and higher concentrations.

As shown in FIG. 6, a calibration curve was prepared to show the relationship between the concentration of p53 DNA concentration and the fluorescence intensity from the magnetic-luminescent nanoassemblies. The inset images are representative molecular assemblies formed at each respective concentration.

### REFERENCES

Each of the following references is incorporated herein by reference in its entirety:
1. Vieira, G., T. Henighan, A. Chen, A. J. Hauser, F. Y. Yang, J. J. Chalmers, and R. Sooryakumar, Magnetic Wire Traps and Programmable Manipulation of Biological Cells. Physical Review Letters, 2009. 103(12): p. 128101.
2. Ruan, G., G. Vieira, T. Henighan, A. Chen, D. Thakur, R. Sooryakumar, and J. O. Winter, Simultaneous Magnetic Manipulation and Fluorescent Tracking of Multiple Individual Hybrid Nanostructures. Nano Letters, 2010. 10(6): p. 2220-2224.
3. Gu, H., R. Zheng, X. Zhang, and B. Xu, Facile One-Pot Synthesis of Bifunctional Heterodimers of Nanoparticles: A Conjugate of Quantum Dot and Magnetic Nanoparticles. Journal of the American Chemical Society, 2004. 126(18): p. 5664-5665.
4. Yi, D. K., S. T. Selvan, S. S. Lee, G. C. Papaefthymiou, D. Kundaliya, and J. Y. Ying, Silica-Coated Nanocomposites of Magnetic Nanoparticles and Quantum Dots. Journal of the American Chemical Society, 2005. 127(14): p. 4990-4991.
5. Deng, S., G. Ruan, N. Han, and J. O. Winter, Interactions in fluorescent-magnetic heterodimer nanocomposites. Nanotechnology, 2010. 21(14): p. 145605.
6. Thakur, D., S. Deng, T. Baldet, and J. O. Winter, pH sensitive CdS-iron oxide fluorescent-magnetic nanocomposites. Nanotechnology, 2009. 20(48): p. 485601.
7. Insin, N., J. B. Tracy, H. Lee, J. P. Zimmer, R. M. Westervelt, and M. G. Bawendi, Incorporation of Iron Oxide Nanoparticles and Quantum Dots into Silica Microspheres. ACS Nano, 2008. 2(2): p. 197-202.
8. Park, J. H., G. von Maltzahn, E. Ruoslahti, S. N. Bhatia, and M. J. Sailor, Micellar hybrid nanoparticles for simultaneous magnetofluorescent imaging and drug delivery. Angew Chem Int Ed Engl, 2008. 47(38): p. 7284-8.
9. Ruan, G., D. Thakur, S. Deng, S. Hawkins, and J. O. Winter, Fluorescent-magnetic nanoparticles for imaging and cell manipulation. Journal of Nanoengineering and Nanosystems, 2010. 223(N3/4): p. 81-86.
10. Ruan, G., D. Thakur, S. Hawkins, and J. O. Winter, Synthesis and manipulation of multifunctional, fluorescent-magnetic nanoparticles for single molecule tracking. Proceedings of SPIE, 2010. 7575.
11. Dubertret, B., P. Skourides, D. J. Norris, V. Noireaux, A. H. Brivanlou, and A. Libchaber, In vivo imaging of quantum dots encapsulated in phospholipid micelles. Science, 2002. 298(5599): p. 1759-1762.
12. Henighan, T., A. Chen, G. Vieira, A. J. Hauser, F. Y. Yang, J. J. Chalmers, and R. Sooryakumar, Manipulation of Magnetically Labeled and Unlabeled Cells with Mobile Magnetic Traps. Biophysical Journal, 2010. 98(3): p. 412-417.
13. McCloskey, K. E., J. J. Chalmers, and M. Zborowski, Magnetophoretic mobilities correlate to antibody binding capacities. Cytometry, 2000. 40(4): p. 307-15.
14. Gratama, J. W., L. D'Hautcourt J, F. Mandy, G. Rothe, D. Barnett, G. Janossy, S. Papa, G. Schmitz, and R. Lenkei, Flow cytometric quantitation of immunofluorescence intensity: problems and perspectives. European Working Group on Clinical Cell Analysis. Cytometry, 1998. 33(2): p. 166-78.
15. Milstein, J. N., M. Chu, K. Raghunathan, and J. C. Meiners, Two-color DNA nanoprobe of intracellular dynamics. Nano Lett, 2012. 12(5): p. 2515-9.
16. Shrivastava, S., J. H. Nuffer, R. W. Siegel, and J. S. Dordick, Position-specific chemical modification and quantitative proteomics disclose protein orientation adsorbed on silica nanoparticles. Nano Lett, 2012. 12(3): p. 1583-7.

What is claimed is:

1. A system for one or more of detecting, isolating, identifying, transporting, and quantifying a target analyte in a fluid suspected of containing the target analyte, the system comprising:
a magnetic nanocomposite comprising one or more selectively magnetic nanoparticles in a first nanocontainer, and a first binding moiety linked to the first nanocontainer and adapted to specifically bind the target analyte; and
a luminescent nanocomposite comprising one or more selectively luminescent nanoparticles in a second nanocontainer, and a second binding moiety linked to the second nanocontainer and adapted to specifically bind the target analyte;
wherein the magnetic nanocomposite and the luminescent nanocomposite do not specifically bind to each other in the absence of the target analyte, and wherein the first and second binding moieties are adapted to simultaneously bind the target analyte to form a magnetic-luminescent nanoassembly.

2. The system of claim 1, wherein the target analyte is a molecular or cellular biomarker.

3. The system of claim 1, wherein the target analyte is soluble in the fluid.

4. The system of claim 1, wherein at least one of the magnetic and fluorescent nanoparticles is insoluble in the fluid.

5. The system of claim 1, wherein the fluid comprises an aqueous solution and at least one of the magnetic and fluorescent nanoparticles is hydrophobic.

6. The system of claim 1, wherein at least one of the first or second nanocontainers comprises an amphiphile.

7. The system of claim 1, wherein at least one of the first and second binding moieties is selected from the group consisting of a peptide, a polypeptide, a protein, a nucleic acid, an oligonucleotide, a polysaccharide, and combinations thereof.

8. The system of claim 1, wherein at least one selectively magnetic nanoparticle comprises superparamagnetic iron oxide.

9. The system of claim 1, wherein at least one selectively luminescent nanoparticle comprises a quantum dot.

10. The system of claim 1, further comprising a magnetic manipulator for generating a magnetic field.

11. The system of claim 10, wherein the magnetic manipulator is selected from the group consisting of a magnetic needle and a magnetic nanoconveyor.

12. A kit comprising the system of claim 1 and instructions for using the system to perform one or more of detecting, isolating, identifying, transporting, and quantifying the target analyte in the fluid.

13. A magnetic-luminescent nanoassembly comprising:
an analyte;
a magnetic nanocomposite comprising one or more selectively magnetic nanoparticles in a first nanocontainer, and a first binding moiety specifically bound to the analyte; and
a luminescent nanocomposite comprising one or more selectively luminescent nanoparticles in a second nanocontainer, and a second binding moiety specifically bound to the analyte;
wherein the magnetic nanocomposite and the luminescent nanocomposite do not specifically bind to each other.

14. A method for one or more of detecting, isolating, identifying, transporting, and quantifying a target analyte in a fluid suspected of containing the target analyte, the method comprising:
adding to the fluid a magnetic nanocomposite comprising one or more selectively magnetic nanoparticles in a first nanocontainer, and a first binding moiety linked to the first nanocontainer and adapted to specifically bind the target analyte;
adding to the fluid a luminescent nanocomposite comprising one or more selectively luminescent nanoparticles in a second nanocontainer, and a second binding moiety linked to the second nanocontainer and adapted to specifically bind the target analyte, wherein the magnetic nanocomposite and the luminescent nanocomposite do not specifically bind to each other in the absence of target analyte, and wherein the first and second binding moieties are adapted to simultaneously bind the target analyte to form a magnetic-luminescent nanoassembly; and
applying a magnetic field to at least a portion of the fluid with a magnetic manipulator.

15. The method of claim 14, further comprising at least one of isolating the target analyte at, or transporting the target analyte to, a location by manipulating the magnetic-luminescent nanoassembly with the magnetic field.

16. The method of claim 14, further comprising at least one of detecting, identifying or quantifying the target analyte by selectively inducing luminescence of the luminescent nanoparticles, and observing at least one of the existence or intensity of luminescence from the magnetic-luminescent nanoassembly.

17. The method of claim 14, further comprises quantifying the target analyte by manipulating the magnetic-luminescent nanoassembly with the magnetic field, and measuring the amount of magnetic mobility.

18. The method of claim 14, wherein at least one of the first or second nanocontainers comprises an amphiphile.

19. The method of claim 14, wherein at least one selectively magnetic nanoparticle comprises superparamagnetic iron oxide.

20. The method of claim 14, wherein at least one selectively luminescent nanoparticle comprises a quantum dot.
